(19) United States
(12) Patent Application Publication
(10) Pub. No.: US 2008/0269540 A1
(43) Pub. Date: Oct. 30, 2008

(54) SEED CARTRIDGE ADAPTOR AND METHODS FOR USE THEREWITH

(75) Inventor: Gary A. Lamoureux, Woodbury, CT (US)

Correspondence Address: FLIESLER MEYER LLP, 650 CALIFORNIA STREET, 14TH FLOOR, SAN FRANCISCO, CA, 94108, US

(73) Assignee: WORLDWIDE MEDICAL TECHNOLOGIES LLC, Oxford, CT (US)

(21) Appl. No.: 12/110,202

(22) Filed: Apr. 25, 2008

Related U.S. Application Data
(60) Provisional application No. 60/914,668, filed on Apr. 27, 2007.

(51) Int. Cl. A61M 36/04

(52) U.S. Cl. 600/7

(57) ABSTRACT

Provided herein are adaptors that enable a seed cartridge that is configured to be used with a first type of needle having a locking hub to instead be used with a second type of needle having a cylindrical hub. Such a seed cartridge has a hub at its distal end that is configured to attach to the locking hub of the first type of needle. The adaptor includes a proximal portion configured to removably attach (and in specific embodiments lock) to the hub at the distal end of the seed cartridge. The adaptor also includes a distal portion configured to removably attach to the cylindrical hub of the second type of needle. A central portion of the adaptor connects the proximal and distal portions of the adaptor. A bore extends through the adaptor and allows seeds and optional spacers to be transferred from the seed cartridge into a hollow cannula of the second type of needle. Also provided herein are methods for using such adaptors.

### PRIORITY CLAIM

This application claims priority under 35 U.S.C. 119(e) to U.S. Provisional Patent Application No. 60/914,668, filed Apr. 27, 2007.

### FIELD OF THE INVENTION

This invention relates to devices and methods that are used for transferring implants to needles used in brachytherapy, and methods for implanting implants.

### BACKGROUND

Brachytherapy is a general term covering medical treatment which involves placement of radioactive sources near a diseased tissue and can involve the temporary or permanent implantation or insertion of radioactive sources into the body of a patient. The radioactive sources are located in proximity to the area of the body which is being treated. A high dose of radiation can thereby be delivered to the treatment site with relatively low doses of radiation to surrounding or intervening healthy tissue. Exemplary radioactive sources include radioactive seeds, radioactive rods and radioactive coils.

Brachytherapy has been used or proposed for use in the treatment of a variety of conditions, including arthritis and cancer. Exemplary cancers that can be treated using brachytherapy include breast, brain, liver and ovarian cancer and especially prostate cancer in men. For a specific example, treatment for prostate cancer can involve the temporary implantation of radioactive sources (e.g., rods) for a calculated period, followed by the subsequent removal of the radioactive sources. Alternatively, radioactive sources (e.g., seeds) can be permanently implanted in the patient and left to decay to an inert state over a predictable time. The use of temporary or permanent implantation depends on the isotope selected and the duration and intensity of treatment required.

Permanent implants for prostate treatment include radioisotopes with relatively short half lives and lower energies relative to temporary seeds. Exemplary permanently implantable sources include iodine-125, palladium-103 or cesium-131 as the radioisotope. The radioisotope can be encapsulated in a biocompatible casing (e.g., a titanium casing) to form a “seed” which is then implanted. Temporary implants for the treatment of prostate cancer may involve iridium-192 as the radioisotope. For temporary implants, radioactive rods are often used.

Conventional radioactive seeds are typically smooth sealed containers or capsules of a biocompatible material, e.g., titanium or stainless steel, containing a radioisotope within the sealed chamber that permits radiation to exit through the container/chamber walls. Other types of implantable radioactive sources for use in radiotherapy are radioactive rods and radioactive coils, as mentioned above.

Preferably, the implantation of radioactive sources for brachytherapy is carried out using minimally-invasive techniques such as, e.g., techniques involving hollow needles. It is possible to calculate a desired location for each radioactive source which will give the desired radiation dose profile. This can be done using knowledge of the radioisotope content of each source, the dimensions of the source, accurate knowledge of the dimensions of the tissue or tissues in relation to which the source is to be placed, plus knowledge of the position of the tissue relative to a reference point. The dimensions of tissues and organs within the body for use in such dosage calculations can be obtained prior to or during placement of the radioactive sources by using conventional diagnostic imaging techniques including X-ray imaging, magnetic resonance imaging (MRI), computed tomography (CT) imaging, fluoroscopy and ultrasound imaging.

During the placement of the radioactive sources into position, a surgeon can monitor the position of tissues such as the prostate gland using, e.g., ultrasound imaging or fluoroscopy techniques which offer the advantage of low risk and convenience to both patient and surgeon. The surgeon can also monitor the position of the relatively large needle used in implantation procedures using ultrasound or other imaging.

As mentioned above, brachytherapy typically employs hollow needles that are insertable into a patient's body, often with the assistance of a template. A typical template used to guide and/or inform the positioning of hollow needles at a surgical site can provide access to more than one hundred locations. The number of locations can be so numerous that a typical pitch between needle access points can include a pitch of 5 mm.

A hollow needle, as explained above, is used to implant radioactive sources and/or other types of treatment elements into patient tissue at a desired location and to a desired depth. Such treatment elements, which are implantable using the hollow needle, shall be collectively referred to as an implant. Such an implant can be an elongate treatment member, such as a strand that includes a plurality of radioactive sources (e.g., seeds) spaced apart from one another within a bioabsorbable material. Besides a strand, an implant can be another type of treatment member that includes a plurality of radioactive sources spaced apart from one another, such as a member formed of seeds and optional spacers that are frictionally or otherwise connected to one another (e.g., as described in U.S. Pat. Nos. 6,010,446 and 6,450,939, which are incorporated herein by reference). An elongate treatment member may also be made from a hollow tube that includes a plurality of seeds and optional spacers loaded within a bore of the tube, with the tube possibly heat shrunk around the seeds and optional spacers, or the ends of the tube otherwise closed. Alternatively, an implant can be a plurality of loose seeds and loose spacers axially aligned one behind the other. It is also possible that the implant be a single loose radioactive source. Other possibilities also exist, as would be appreciated by one of ordinary skill in the art. For example, an implant can include one or more radioactive rod or coil. An implant can also include one or more seed that has anchoring mechanisms, exemplary details of which are provided in commonly assigned U.S. patent application Ser. No. 11/187,411, entitled “Implants for Use in Brachytherapy and Other Radiation Therapy That Resist Migration and Rotation,” filed Jul. 22, 2005. Alternatively, the implant can be or include some other object and need not be radioactive, e.g. a spacer, a marker, or a thermal seed that gives off heat.

FIG. 1 is a perspective view of an exemplary seed cartridge assembly 11 that can be used for brachytherapy. The seed cartridge assembly 11 is adapted to hold and dispense radioactive seeds which may be employed in the treatment of, for example, cancerous prostates. In FIG. 2, the seed cartridge assembly 11 is fully assembled and includes a seed cartridge 15 and a radiation shield 10. The seed cartridge 15 includes a cartridge body 14 and a seed drawer 16.

FIG. 2 is an exploded perspective view of the elements of the seed cartridge assembly 11 of FIG. 1, including the seed cartridge 15 and the radiation shield 10. The cartridge body 14 of seed cartridge 15 includes a cartridge hub 28 and a cartridge shaft 29. The cartridge hub 28 includes an upper needle guide 26, cartridge hub grips 32, hub locking flanges 44, a luer opening 78 and an orientation indicator 90. The cartridge shaft 29 includes a viewing lens 30, a distal shield locking rib 46, an intermediate shield locking rib 50 and a proximal shield locking rib 52. The viewing lens 30 may be, for example, a prism. The seed drawer 16 of the seed cartridge 15 includes locking cylinder 22, vents 24, lower needle guide 54, lower locking recess 56, locking spring 58, rear handle 60, seed channel 64, locking nib 66 and seed retainer 74. In FIG. 2, the brachytherapy seeds 20 are interspersed with spacers 18. The spacers 18 may be, for example, absorbable spacers made from an autoclaveable material such as, for example a polyglectin 910, but are not limited thereto. With the seed drawer 16 positioned in its closed position, the upper needle guide 26 and the lower needle guide 54 combine to form a needle guide 27. The radiation shield 10 of the seed cartridge assembly 11 includes a locking tab 12.

Within the seed drawer 16 is nestled a seed retainer 74 which is adapted to passively enclose the brachytherapy seeds 20 and spacers 18 in the seed channel 64 until the seeds 20 and the spacers 18 are propelled through seed channel 64 and out needle guide 27 by, for example, a stylet 84 (see FIG. 3). Still referring to FIG. 2, the seed cartridge assembly 11 further includes a luer opening 78 which is adapted to mate with a conventional brachytherapy needle 82 (also known as a locking hub needle, or a luer lock needle) having a conventional luer mating element (e.g., a peripheral flange 93 in FIG. 3) or threads (e.g., 114 in FIG. 5B). An alternative form factor of a hub for a locking hub needle is shown in FIG. 5B discussed below.

FIGS. 3 and 4 illustrate how seeds 20 are incorporated into a brachytherapy needle 82. FIG. 3 is an exploded perspective view of the seed cartridge assembly 11, brachytherapy needle 82, and a stylet 84. In the embodiment illustrated, the brachytherapy needle 82 can be used, e.g., in brachytherapy procedures involving treatment of cancer of the prostate.

In the embodiment illustrated, a locking needle hub 88 of the brachytherapy needle 82 may be attached to the cartridge hub 28 (of the seed cartridge 15) with a turn (e.g., a sixty degree turn), locking the proximal end of brachytherapy needle 82 to the distal end of seed cartridge assembly 11. The stylet 84 may then be used to move spacers 18 and seeds 20 from seed cartridge assembly 11 to a needle cannula 86. Once the spacers 18 and seeds 20 are positioned in the needle cannula 86, the stylet 84 may be removed. The seed cartridge assembly 11 is then disconnected from brachytherapy needle 82, and the stylet 84 is positioned in brachytherapy needle 82 to be used intraoperatively as in a normal brachytherapy procedure utilizing preloaded needles. FIG. 4 is a perspective view of a loaded brachytherapy needle 82 with the seed cartridge assembly 11 removed and the stylet 84 inserted into the needle hub 88. The needle 82 that is preloaded using the seed cartridge assembly 11 (which has since been disconnected from the needle 82) can then be inserted into patient tissue. Once the brachytherapy needle 82 is properly positioned within the patient, the stylet 84 may be used to force the seeds 20 and spacers 18 out of the needle cannula 86 and into the portion of the patient to be treated, such as, for example, the prostate.

Additional details of the exemplary seed cartridge assembly 11 are disclosed in U.S. Pat. No. 6,585,633, which in incorporated herein by reference.

### SUMMARY OF EMBODIMENTS OF THE INVENTION

Embodiments of the present invention relate to adaptors that enables a seed cartridge that is configured to be used with a first type of needle having a locking hub to instead be used with a second type of needle having a cylindrical hub. Such a seed cartridge has a hub at its distal end that is configured to attach to the locking hub of the first type of needle. The adaptor includes a proximal portion configured to removably attach (e.g., removably lock or friction fit) to the hub at the distal end of the seed cartridge. The adaptor also includes a distal portion configured to removably attach to the non-locking cylindrical hub of the second type of needle. A central portion of the adaptor connects the proximal and distal portions of the adaptor. A bore extends through the adaptor and allows seeds and optional spacers to be transferred from the seed cartridge into a hollow cannula of the second type of needle.

Embodiments of the present invention are also related methods of implanting seeds and optional spacers into patient tissue using a needle having a cylindrical hub, an adaptor, and a seed cartridge that is configured to be used with a needle having a locking hub.

This summary is not intended to be a complete description of the invention. Other features, aspects, objects and advantages of the invention can be obtained from a review of the specification, the figures, and the claims.

### BRIEF DESCRIPTION OF THE DRAWINGS

FIG. 1 is a perspective view of a prior art seed cartridge assembly that can be used for brachytherapy.

FIG. 2 is an exploded perspective view of the elements of the seed cartridge assembly of FIG. 1.

FIG. 3 is an exploded perspective view of the seed cartridge assembly of FIGS. 1 and 2 and a brachytherapy needle and a stylet.

FIG. 4 is an perspective view of a loaded brachytherapy needle with the seed cartridge assembly of FIGS. 1-3 removed and the stylet of FIG. 3 inserted into needle hub of the needle.

FIG. 5A is a side view of first type of hollow brachytherapy needle, often referred to as an applicator needle.

FIG. 5B is a side view of a second type of hollow brachytherapy needle, sometimes referred to as a locking hub needle or a luer lock needle.

FIG. 6A is a perspective view of an adaptor that enables the prior art seed cartridge assembly of FIGS. 1-4 to be used with the hollow applicator needle shown in FIG. 5A.

FIG. 6B is a cross sectional view of the adaptor of FIG. 6A.

FIG. 7 is a cross section view of the adaptor having an alternative form factor, in accordance with an embodiment.

FIG. 8 is a high level flow diagram that is used to summarize a method of using the adaptor of FIGS. 6A, 6B and 7, in accordance with an embodiment of the present invention.

FIG. 9 is a high level flow diagram that is used to summarize a method of using the adaptor of FIGS. 6A, 6B and 7, in accordance with an alternative embodiment of the present invention.

### DETAILED DESCRIPTION

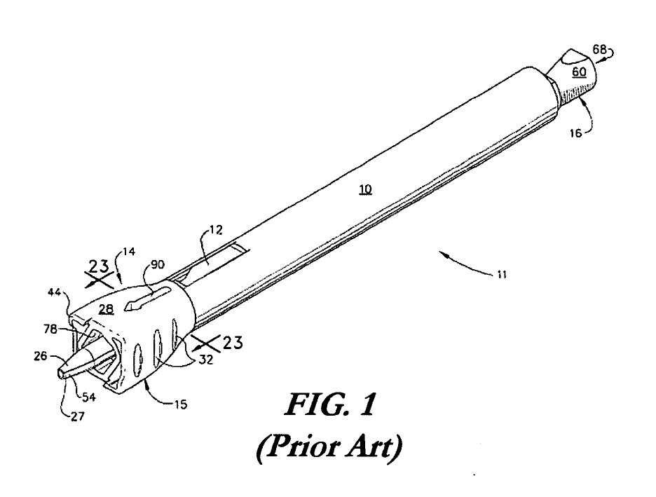

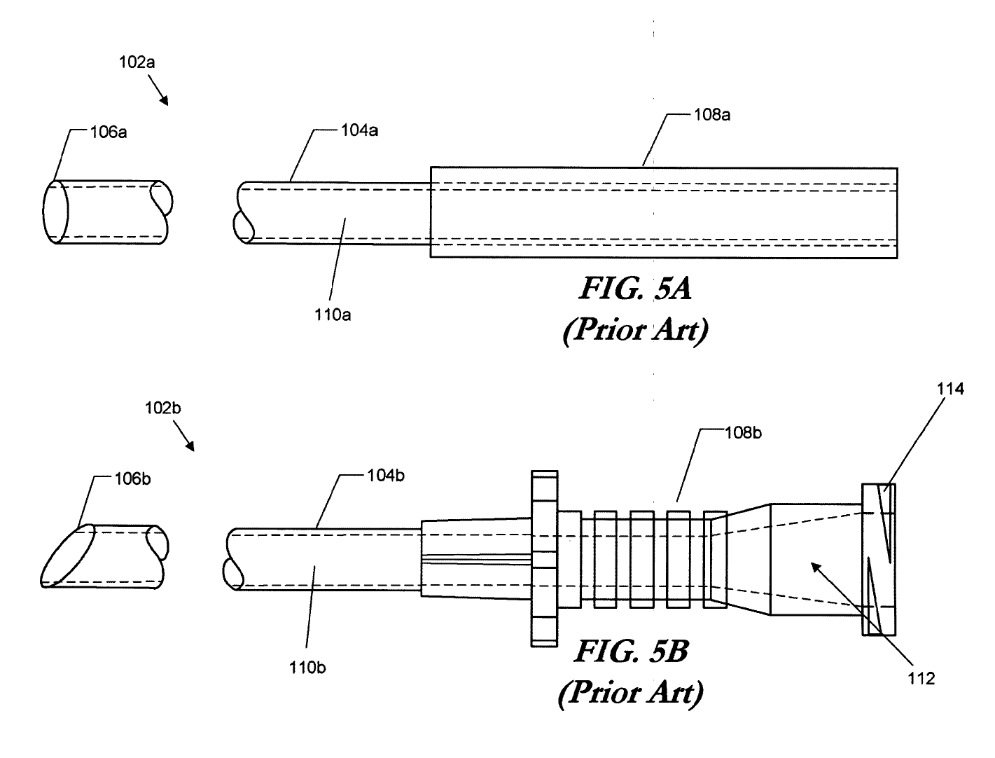

Various types of hollow needles can be used in brachytherapy, examples of which are shown in FIGS. 5A and 5B. A first type of needle, shown in FIG. 5A, is often referred to as an applicator needle, and is sometimes marketed under the trademark MICK® needle. Referring to FIG. 5A, an applicator needle 102a is shown as including a hollow needle 104a (also referred to as a cannula) with a blunt or un-sharpened distal end 106a, and a hub 108a positioned at a proximal end. The hub 108a, as shown, has a generally simple cylindrical shape that does not include threads or a peripheral flange. Accordingly, the hub 108a may be referred to as a non-locking hub. An exemplary length of the entire needle 102a including the hub is about 7⅞ inches (about 20 cm), with the hub 108a having a length of about 1 inch (about 2.5 cm). As shown in FIG. 1A, the hub 108a surrounds a proximal portion of the cannula 104a. A bore 110a (also referred to as a lumen) extends through the applicator needle 102a. An exemplary diameter of the bore 110a (i.e., the inner diameter of the cannula 104a) is about 0.042 inches.

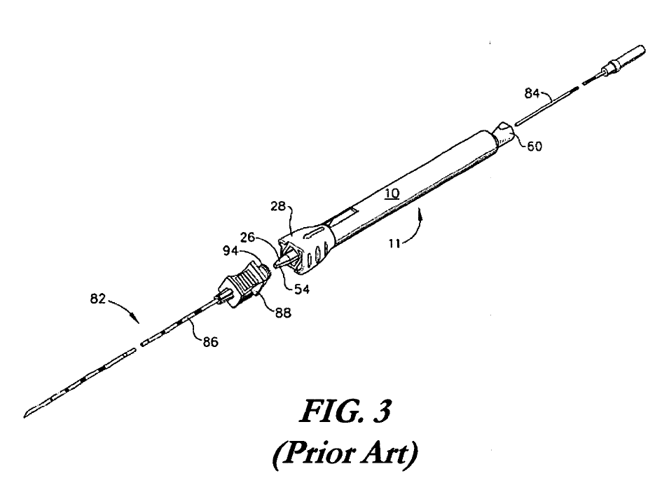

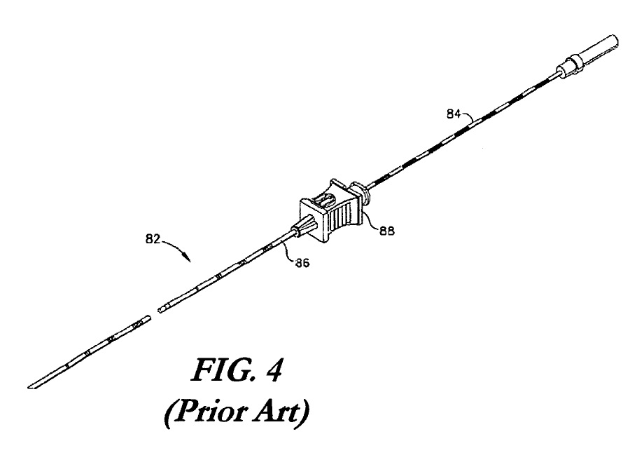

When an applicator needle 102a is used in brachytherapy, a sharp stylet (not shown in FIG. 5A) is inserted through the lumen 110a of the hollow needle, so that the sharp distal end of the sharp stylet (e.g., a trocar tip) extends past the blunt distal end 106a of the applicator needle 102a. The needle 102a, with the sharp stylet point extending out its blunt distal end 106a, can then be inserted into patient tissue at a desired location, including to a desired depth. Thereafter, the sharp stylet is removed, and an implant (e.g., a strand, seeds and optional spacers, or combinations thereof) is loaded into the needle through the proximal end of the needle. Tweezers or the like have been used to insert a strand or other treatment member, and/or loose seeds and optional spacers, into the proximal end of the needle. However, this can be very difficult and time consuming due to the small inner diameter of the hollow needle and the small size of the implant. A blunt ended stylet (not shown in FIG. 5A, but similar to the stylet 84 shown in FIGS. 3 and 4) can then be inserted into the proximal opening of the needle 102a, until the distal end of the stylet contacts the proximal end of the treatment member (or most proximal seed or spacer). The needle can then be retracted with the stylet held in position so that the implant is deposited at a desired location.

Alternatively, an applicator device, such as a MICK® applicator, can be attached to the proximal end of the applicator needle 102a, and the applicator device can be used to dispose loose seeds (and optionally loose spacers) through the needle 102a and into patient tissue. The MICK® applicator is available from Mick Radio-Nuclear Instruments, Inc., Mount Vernon, N.Y. Exemplary details of the MICK® applicator are provided in U.S. Pat. No. 5,860,909.

Referring now to FIG. 5B, a second type of needle 102b, which shall be referred to herein as a locking hub needle for reasons that will be apparent (and sometimes referred to as a prostate seed needle, a standard needle, or a luer lock needle), includes a cannula 104b, a sharpened distal end 106b (e.g., a beveled end), and a hub 108b positioned at a proximal end. The hub 108b has an enlarged diameter with a funneled proximal portion 112, and threads 114 or a peripheral flange to provide a male luer connector on an outer circumference, that may be used, e.g., to connect the hub 108b to a syringe. The brachytherapy needle 82, discussed above, and shown in FIGS. 3 and 4, is another example of a type of locking hub needle. The hub 88 of the needle 82 (shown in FIGS. 3 and 4) is an alternative form factor of the hub 108b. Other form-factors for the locking hub besides those shown in FIGS. 3, 4 and 5B are also possible.

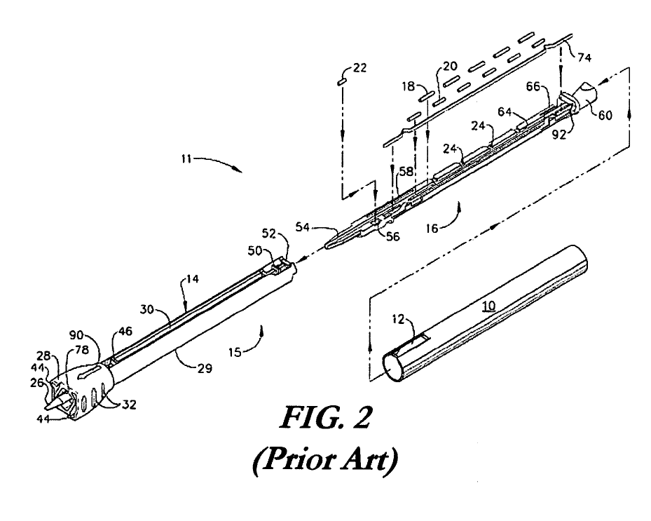

The seed cartridge assembly 11 described with reference to FIGS. 1-4 is configured to load seeds and optional spacers into a locking hub needle (e.g., need 100b shown in FIG. 5B, or need 82 shown in FIGS. 3 and 4). A problem with the seed cartridge assembly 11, and more specifically the seed cartridge 15, is that it can only be used with a locking hub needle (e.g., 102b or 82), but it can not be used with an applicator needle, such as the applicator needle 102a shown in FIG. 5A. Some physicians prefer using an applicator needle rather than a locking hub needle.

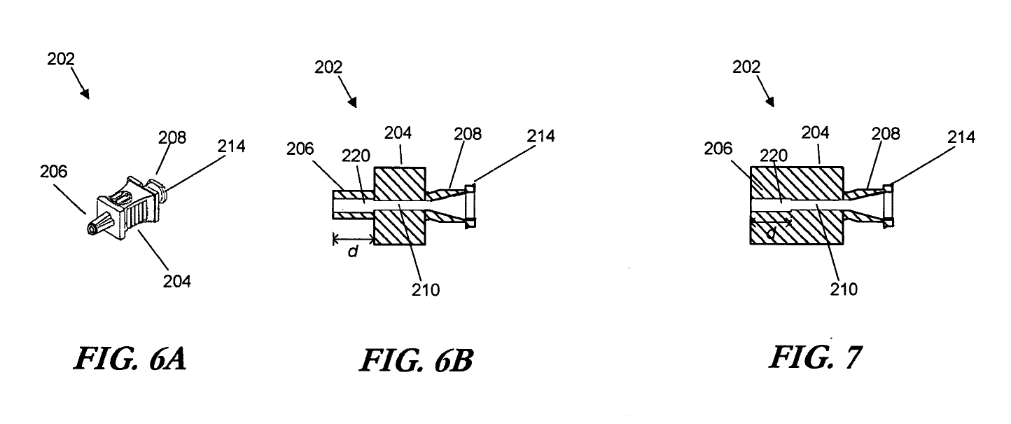

Provided in FIGS. 6A and 6B is an adaptor 202 that enables the seed cartridge assembly 11 (and more generally, the seed cartridge 15) to be used with an applicator needle 102a, which has a simple cylindrical hub 108a. While it is preferred that the seed cartridge 11 be used with the radiation shield 10, that is not required, and embodiments of the present invention should not be limited to use with the shield 10.

FIG. 6A is a perspective view of the adaptor 202, according to an embodiment of the present invention. FIG. 6B is a cross-section of the adaptor 202. The adaptor 202 includes a central portion 204, from which extends a distal portion 206 and a proximal portion 208. A bore 210 extends axially through the entire adaptor 202. The diameter of the bore 210 should be sufficient to allow an implant (e.g., seeds and optional spacers) to pass there-through. Thus, the diameter of the bore 210, or a portion thereof, can be similar to the diameter of the bores 110a and 110b of needles 102a and 102b with the inner diameter being about the same as the inner diameter of the needle with which the adaptor 202 will interface (e.g., the inner diameter of the cannula of the hollow needles 102a and 102b can be about 0.042 inches).

The central portion 204 and proximal portion 208 of the adaptor 202 can be similar to the needle hub 88 shown in FIGS. 3 and 4, or the needle hub 108b shown in FIG. 5B, as can be appreciated from FIGS. 6A and 6B. This enables the adaptor 202 to attach to the cartridge hub 28 at the distal end of the seed cartridge 15. Other form factors of the central portion (e.g., a cylindrical outer shape in place of the rectangular outer shape shown) are also possible, and within the scope of embodiments of the present invention. The proximal portion 208 includes threads or a peripheral luer lock flange (collectively designated 214) to allow for connection to female threads or a female luer connector (e.g., 78) of the hub 28 of the seed cartridge 15. In other words, the proximal portion 208 is configured to be to removably lock to the hub 28 at the distal end of the seed cartridge 15. The proximal portion 208 can alternatively removable attach to the hub 28 of the seed cartridge 15 using a friction fit, or some alternative removable attachment mechanism.

In accordance with an embodiment, the inner diameter of the distal portion 206 of the adaptor 202 is larger than the inner diameter of the central portion 204 of the adaptor 202, so that the hub 108a of the applicator needle 102a can be accepted therein. In other words, the diameter of the distal portion 206 of the bore 210, designated 220 (which is the portion of the bore 210 within distal portion 206), is larger than the diameter of the central portion of the bore 210 (i.e., the portion of the bore 210 extending through the central portion 204 of the adaptor 204). More specifically, the distal opening of the portion 220 of the bore 210, extending from the distal end 206 towards the central portion 204 of the adaptor 202, has a diameter that is slightly larger than the outer diameter of the hub 108a of the applicator needle 102a, so that the distal portion 220 of the bore 210 is configured to receive, and thereby connect to, the hub 108a of the applicator needle 102a. For example, the diameter of the distal portion 220 of the bore 210 can be 0.099 inches, while the diameter of the main portion of the bore 210, which roughly corresponds to the inner diameter of a hollow needle, can be anywhere from about 0.042 to 0.055 inches. In specific embodiments, the distal portion 220 of the bore 210 has a slight taper from its opening rearward (e.g., an opening diameter of 0.105 inches tapers to 0.985 inches), so that a slight friction fit can be provided between the distal portion 220 of the bore 210 and the hub 108a accepted therein (presuming the outer diameter of the hub 108a is less than 0.105 inches, but greater than 0.985 inches).

Additionally, a depth (d in FIG. 6B) of the distal portion 220 of the bore 210 is preferably sufficient to enable the adaptor 202 to rigidly connect to the hub 108a of the applicator needle 102a without requiring any additional support, e.g., from a user or some support structure (e.g., a support rod). Preferably such depth d is at least ¼ inch, and more preferably about ½ inch. However, other depths will work, and are within the scope of the present invention.

FIG. 7 shows an alternative form factor for the adaptor 202. Many other form factors are also possible, and within the scope of the present invention.

As explained above, the adaptor 202 can be used to transfer seeds and optional spacers from the seed cartridge assembly 11 to an applicator needle 102a, after such needle 102a has been inserted into patient tissue. As mentioned above, since the applicator needle 102a has a blunt distal end 106a, a sharp ended stylet would likely be used to assist with insertion of the needle 102a into patient tissue. The sharp stylet would then be removed, and the adaptor 202 can be used to transfer seeds and optional spacers from the seed cartridge assembly 11 to the needle 102a, e.g., using a blunt ended stylet, or alternatively using the same sharp ended stylet if so desired.

More specifically, a distal end of a hollow needle 102a can be implanted into patient tissue at a desired location and to a desired depth, where the needle 102a has a simple cylindrical needle hub 108a at its proximal end (i.e., a hub that does not include threads or a peripheral flange, as was the case with hub 88 and 108b). The adaptor 202 is used to attach the cylindrical needle hub 108a to a distal end of the seed cartridge 15, where as explained above, the hub 28 at the distal end of the seed cartridge is intended to be attached to a threaded or luer locking needle hub (e.g., 88 or 108b). A stylet is inserted into an opening at a proximal end of the seed cartridge assembly 11. Seeds and optional spacers are urged from the seed cartridge assembly 11, through the bore 210 of the adaptor 202, and through a lumen 110a of the hollow needle 102a, to a distal end of the hollow needle 102a. If the stylet is long enough, the hollow needle 108a and the seed cartridge assembly 11 can be retracted, while the stylet is held in place, to thereby deposit the seeds and optional spacers at the desired location and to the depth. Alternatively, if the style is not long enough, the stylet is retracted from the seed cartridge assembly 11, and the seed cartridge assembly 11 (and likely but not necessarily the adaptor 202) is/are removed. Thereafter, a stylet is inserted (e.g., reinserted) into an opening at the proximal end of the hollow needle 108a, and the seeds and optional spacers are urged to a distal end of the hollow needle. The hollow needle is then retracted, while the stylet is held in place, to thereby deposit the seeds and optional spacers at the desired location and to the desired depth.

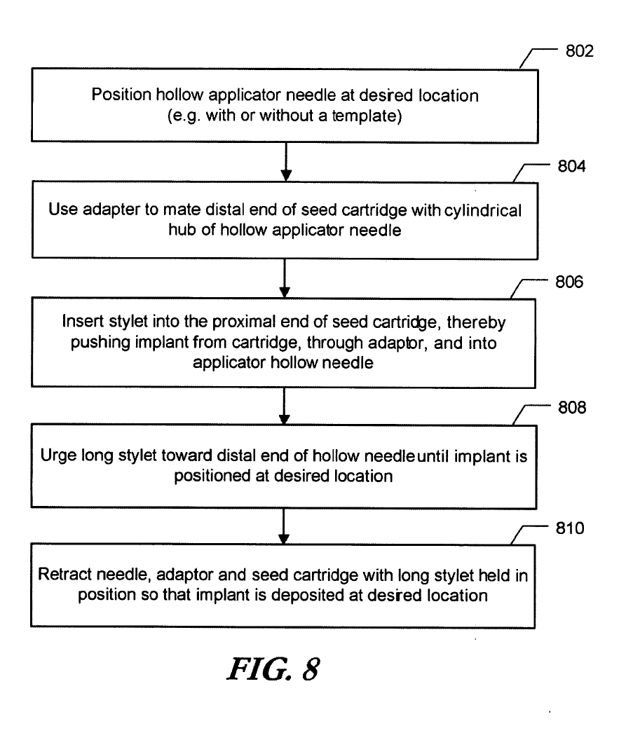

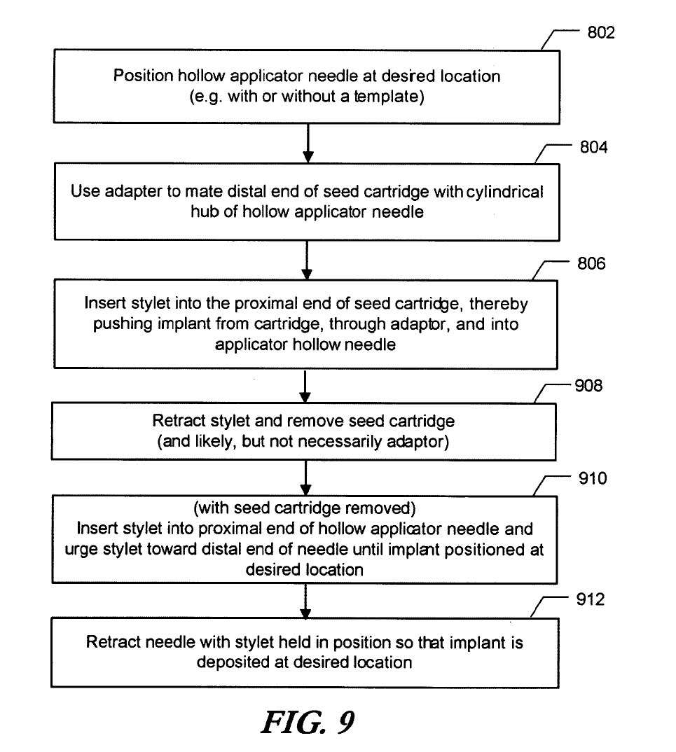

Methods for using the adaptor 202 shall now be summarized with reference to FIGS. 8 and 9. Steps that are common to each method are numbered in the same manner, to avoid replication of the discussion.

Referring to FIG. 8, at step 802, the hollow applicator needle 102a is positioned at the desired location, e.g., with the assistance of a template and likely a sharp ended stylet. At step 804, the adaptor 202 is used to mate the hub 108a of the hollow applicator needle 102a with the distal end of the seed cartridge assembly 11 (and more specifically, the hub 28 of the seed cartridge 15). At step 806, with the implant (e.g., seeds and optional spacers) positioned within the cartridge assembly 11, and the cartridge assembly connected to the hub 108a of the hollow applicator needle 102a needle using the adaptor 202, a stylet is inserted into the opening at the proximal end of the seed cartridge assembly 11. The stylet can be sufficient in length to accommodate both the needle 102a, the adaptor 202 and the cartridge assembly 11, as well as the depth to which the implant is to be deposited. Where that is the case, at step 808, the implant is urged toward the distal end of the needle until the implant is positioned at the desired location and depth. Then, the stylet is held in place while the needle, adaptor and cartridge are retracted, so that the implant is deposited at the desired location to the desired depth, as indicated at step 810. Alternatively, a stylet of less than sufficient length (to accommodate both the needle 102a, the adaptor 202, the seed cartridge assembly 11 and the depth to which the implant is to be deposited) can be employed. When such a shorter stylet is used, the stylet can be used urge the implant from the seed cartridge assembly 11 through the adaptor 202 into to the hollow needle 102a, as indicated at step 806. The stylet can then be removed, and the cartridge 11 (and likely but not necessarily the adaptor 202) can be disconnected from the needle hub 108a, as indicated at step 908 in FIG. 9. A stylet (the same, or a different stylet) can then be inserted (e.g., reinserted) to urge the implant to the desired location and depth, at step 910. At step 912, the needle 102a can then be retracted with the stylet held in position so that the implant is deposited at the desired location and depth.

While the adaptor 202 was described as being used with the exemplary seed cartridge assembly 11 shown and described with reference to FIGS. 1-4, the adaptor 202 can also be used with alternative seed cartridges that are intended to attach to a locking hub needle, but for which a physician would prefer to use a simple applicator needle 102a having a cylindrical hub without threads or a peripheral flange.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. An adaptor that enables a seed cartridge that is configured to be used with a first type of needle having a locking hub to instead be used with a second type of needle having a cylindrical hub, wherein the seed cartridge has a hub at its distal end that is configured to attach to the locking hub of the first type of needle, the adaptor including:
a proximal portion configured to removably attach to the hub at the distal end of the seed cartridge;
a distal portion configured to removably attach to the cylindrical hub of the second type of needle;
a central portion that connects said proximal and distal portions; and
a bore extending through said proximal, central and distal portions, and that allows seeds and optional spacers to be transferred from the seed cartridge into a hollow cannula of the second type of needle.

2. The adaptor of claim 1, wherein the proximal portion is configured to removably lock to the hub at the distal end of the seed cartridge.

3. The adaptor of claim 1, wherein the proximal portion is configured to removably friction fit to the hub at the distal end of the seed cartridge.

4. The adaptor of claim 1, wherein:
a distal portion of said bore has a first diameter that is slightly larger than the outer diameter of the cylindrical hub of the second type of needle thereby enabling the cylindrical hub of the second type of needle to be removably accepted in said distal portion of said bore; and
a main portion of said bore has a second diameter that as smaller than the outer diameter of the cylindrical hub of the second type of needle to thereby prevent the cylindrical hub of the second type of needle from being accepted in said main portion of said bore.

5. The adaptor of claim 4, wherein:
said second diameter of said main portion of said bore is approximately equal to the inner diameter of the hollow cannula of the second type of needle.

6. The adaptor of claim 4, wherein:
a depth of said distal portion of said bore is sufficient to enable the adaptor to rigidly connect to the cylindrical hub of the second type of needle without requiring additional support.

7. The adaptor of claim 1, wherein the seed cartridge includes a channel for storing seeds and optional spacers, and wherein:
said bore of the adaptor provides a continuous channel between the channel of the seed cartridge that stores seeds and optional spacers and the hollow cannula of the second type of needle.

8. The adaptor of claim 1, wherein said proximal portion of the adaptor includes peripheral threads and/or a peripheral flange that enables said proximal portion to removably lock to the hub at the distal end of the seed cartridge.

9. An adaptor that enables a seed cartridge that is configured to be used with a first type of needle having a locking hub to instead be used with a second type of needle having a cylindrical hub, wherein the seed cartridge has a hub at its distal end that is configured to attach to the locking hub of the first type of needle, the adaptor including:
a first portion configured to removably attach to the hub at the distal end of the seed cartridge;
a second portion configured to removably attach to the cylindrical hub of the second type of needle; and
a bore extending through said first and second portions, said bore to allow seeds and optional spacers to be transferred from the seed cartridge into a hollow cannula of the second type of needle.

10. The adaptor of claim 9, wherein said first portion of the adaptor includes peripheral threads or a peripheral flange that enables said first portion to removably lock to the hub at the distal end of the seed cartridge.

11. The adaptor of claim 9, wherein said first portion of the adaptor is configured to removably friction fit to the hub at the distal end of the seed cartridge.

12. The adaptor of claim 9, wherein:
a first portion of said bore has a first diameter that is slightly larger than the outer diameter of the cylindrical hub of the second type of needle to thereby enable the cylindrical hub of the second type of needle to be removably accepted in said first portion of said bore; and
a second portion of said bore has a second diameter that is smaller than the outer diameter of the cylindrical hub of the second type of needle to thereby prevent the cylindrical hub of the second type of needle from being accepted in said second portion of said bore.

13. The adaptor of claim 12, wherein:
a depth of said first portion of said bore is sufficient to enable the adaptor to rigidly connect to the cylindrical hub of the second type of needle without requiring additional support.

14. The adaptor of claim 9, wherein the seed cartridge includes a channel for storing seeds and optional spacers, and wherein:
said bore of the adaptor provides a continuous channel between the channel of the seed cartridge that stores seeds and optional spacers and the hollow cannula of the second type of needle.

15. A method for implanting seeds and optional spacers into patient tissue, the method comprising:
(a) inserting a distal end of a hollow needle into patient tissue at a desired location and to a desired depth, the needle having a cylindrical needle hub at its proximal end;
(b) using an adaptor to attach the cylindrical needle hub to a proximal end of a seed cartridge;
(c) inserting a stylet into an opening at a proximal end of the seed cartridge;
(d) urging seeds and optional spacers from the seed cartridge assembly, through the adaptor, and through a lumen of the hollow needle, to a distal end of the hollow needle; and
(e) retracting the hollow needle and the seed cartridge, while the stylet is held in place, to thereby deposit the seeds and optional spacers at the desired location and to the depth.

16. The method of claim 15, wherein step (b) includes:
(b.1) connecting the adaptor to the cylindrical needle hub of the hollow needle by inserting at least a portion of the cylindrical needle hub into a distal portion of a bore of the adaptor, such that the adaptor is rigidly connected to the cylindrical needle hub of the hollow needle without requiring additional support; and
(b.2) connecting the seed cartridge to a proximal end of the adaptor.

17. A method for implanting seeds and optional spacers into patient tissue, the method comprising:
(a) inserting a distal end of a hollow needle into patient tissue at a desired location and to a desired depth, the needle having a cylindrical needle hub at its proximal end;
(b) using an adaptor to attach the cylindrical needle hub to a proximal end of a seed cartridge assembly;
(c) inserting a stylet into an opening at a proximal end of the seed cartridge;
(d) urging seeds and optional spacers from the seed cartridge, through the adaptor, and through a lumen of the hollow needle, to a distal end of the hollow needle;
(e) retracting the stylet from the seed cartridge;
(f) removing the seed cartridge and optionally the adaptor;
(g) inserting a stylet into an opening at a distal end of the hollow needle;
(h) urging the seeds and optional spacers to a distal end of the hollow needle; and
(i) retracting the hollow needle, while the stylet is held in place, to thereby deposit the seeds and optional spacers at the desired location and to the depth.

18. The method of claim 17, wherein step (b) includes:
(b.1) connecting the adaptor to the cylindrical needle hub of the hollow needle by inserting at least a portion of the cylindrical needle hub into a distal portion of a bore of the adaptor, such that the adaptor is rigidly connected to the cylindrical needle hub of the hollow needle without requiring additional support; and
(b.2) connecting the seed cartridge to a proximal end of the adaptor.
